(19) United States
(12) Patent Application Publication
(10) Pub. No.: US 2009/0270926 A1
(43) Pub. Date: Oct. 29, 2009

(54) ROTOLOCK CERVICAL PLATE LOCKING MECHANISM

(76) Inventor: David T. Hawkes, Pleasant Grove, UT (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 12/429,926

(22) Filed: Apr. 24, 2009

Related U.S. Application Data
(60) Provisional application No. 61/047,684, filed on Apr. 24, 2008.

(51) Int. Cl. A61B 17/80; A61B 17/86

(52) U.S. Cl. 606/286; 606/301

(57) ABSTRACT

An orthopedic plate contains a thru-bore with an oval shaped rim and an internal groove underneath that rim. A compressible retention member is designed with an upper plane and a lower plane. The upper plane being oval shaped and smaller in diameter than the lower plane. The lower plane is designed to extend into the internal groove cut into the orthopedic device. After the screw has been inserted, the compressible retention member is rotated, squeezing the larger dimension of the oval shaped ring into the smaller dimension of the oval shaped rim. This reduces the diameter of the compressible retention member. The compressible retention member has an overhang section that when compressed, covers up part of the top edge of the screw to prevent it from backing out.

### RELATED APPLICATIONS

This application claims the benefit under 35 U.S.C. § 119(e) of U.S. Provisional Patent Application No. 61/047,684 filed Apr. 24, 2008 titled “Rotationally Activated Oval Locking Ring and Undercut Screw Feature,” which provisional application is incorporated herein by reference in its entirety.

### FIELD

The present system and method relate to bone fixation devices. More particularly, the present system and method provide for an orthopedic system including a plate, a screw system, and a complete system including the plate system, the screw system, and the screw retention system.

### BACKGROUND

In the treatment of various spinal conditions, including the treatment of fractures, tumors, and degenerative conditions, it is necessary to secure and stabilize the spine following removal of a vertebral body or part. Various devices for internal fixation of bone segments in the human or animal body are known in the art.

Following such removal made using a thoracotomy, thoracoabdominal or retroperitoneal approach, the normal anatomy is reconstructed using tricortical iliac crest or fibular strut grafts. Not only are removals performed on the thoracic spine, as is the case for the above procedures, but also the cervical spine. Once bone matter is removed, it is then necessary to secure and stabilize the graft, desirably in such a manner as to permit rapid mobilization of the patient. Such objectives can be accomplished by a bone plate. However, to accomplish this service in the optimum manner, it is necessary that the plate be reasonably congruent with the bone to which it is applied, that it have as low a profile as possible, that it be firmly secured to the spinal column so that it is not torn out when the patient places weight and stress upon it and that it be capable of placement and fixation in a manner that is convenient for the surgeon.

In this context it is necessary to secure the plate to the spinal body and also, in some cases, to the graft. Conventionally, such attachment would be by the use of screws driven through screw holes in the plate into the bone. However, when stabilizing the position of cervical vertebrae, the plate is designed to lie near and posterior to the esophagus of the patient. Due to its relative location to the esophagus and other connective tissue, if the screw securing the plate to the cervical spine backs out, the screw could irritate or even pierce the esophagus, resulting in pain, infection, and/or possible death of the patient. Consequently, anti-back out mechanisms are desired in the orthopedic plate industry.

### SUMMARY

According to one exemplary embodiment, an orthopedic bone fixation device for stabilizing a plurality of bone segments includes a bone plate and a screw assembly. The bone plate includes a body defining at least one thru-bore, wherein the thru-bore is defined to include a central cavity, the central cavity includes a split ring, a compliant member, or another positionable element configured to modify an exit diameter of the thru-bore. Additionally, an actuation member is coupled to the bone plate. According to one exemplary embodiment, actuation of the actuation member, either by rotation, sliding, or the like, causes the actuation member to engage the positionable member, thereby modifying the exit diameter of the thru-bore. Further, the screw assembly is configured to be coupled to the bone plate, wherein the screw assembly includes a bone screw having a head section and a thread section. When actuated, the positionable element is configured to reduce the exit diameter of the thru-bore sufficient to interfere with the head section of the bone screw, thereby preventing the screw from backing out.

### BRIEF DESCRIPTION OF THE DRAWINGS

The accompanying drawings illustrate various exemplary embodiments of the present system and method and are a part of the specification. Together with the following description, the drawings demonstrate and explain the principles of the present system and method. The illustrated embodiments are examples of the present system and method and do not limit the scope thereof.

FIG. 1 is an illustrative depiction of a top view of an exemplary orthopedic plate with a thru-bore in the middle, according to one embodiment of principles described herein.

FIG. 2 is an illustrative depiction of a side view of an exemplary thru-bore in an orthopedic plate, according to one embodiment of principles described herein.

FIG. 3A is an illustrative depiction of a top view of an exemplary positionable element, according to one embodiment of principles described herein.

FIG. 3B is an illustrative depiction of a side view of an exemplary positionable element, according to one embodiment of principles described herein.

FIG. 4A is an illustrative depiction of a top view of an exemplary orthopedic plate with an exemplary positionable element inside the thru-bore, according to one embodiment of principles described herein.

FIG. 4B is an illustrative depiction of a side view of an exemplary orthopedic plate with an exemplary positionable element and screw inside the thru-bore, according to one embodiment of principles described herein.

FIG. 5A is an illustrative depiction of a top view of an exemplary orthopedic plate with an exemplary secured positionable element and screw inside the thru-bore, according to one embodiment of principles described herein.

FIG. 5B is an illustrative depiction of a side view of an exemplary orthopedic plate with an exemplary secured positionable element and screw inside the thru-bore, according to one embodiment of principles described herein.

FIG. 6 is an illustrative depiction of an exemplary screw head with a radial groove configured to receive a compressible retention ring, according to one embodiment of principles described herein.

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings. Throughout the drawings, identical reference numbers designate similar but not necessarily identical elements.

### DETAILED DESCRIPTION

The present specification describes a system and a method for preventing screws used in orthopedic devices from backing out. According to one exemplary embodiment, an oval shaped compressible retention member, such as a split-ring is placed inside an oval shaped slot on an orthopedic plate where a screw is to be inserted. Once the screw has been driven into place, the positionable element may be rotated to reduce the diameter of its inner edge, thereby covering the screw and preventing it from backing out of the orthopedic plate. As used herein, for ease of explanation only, the present system and method will be described in terms of a compression occurring from the selective rotation of an oval compressible member within an oval orifice. However, it will be understood that the present exemplary system and method may be performed by the rotation of a compressible retention member having a non-circular perimeter in a non-circular orifice.

By way of example, orthopedic plate systems may be used in the treatment of various spinal conditions. As mentioned, when applied to stabilize the position of cervical vertebrae, the plate portion of the orthopedic plate system is designed to lie near and posterior to the esophagus of the patient. Due to its relative location to the esophagus and other connective tissue, the top surface of the plate portion may be smooth and free of sharp corners to prevent irritation or piercing of the esophagus and surrounding tissue. Further, in order to prevent irritation and/or piercing, any connection hardware that is used to couple the plate portion to the cervical vertebrae should remain below or even with the top surface of the plate portion.

If the screw or other fastener securing the plate portion to the cervical spine backs out or otherwise protrudes above the top surface of the plate portion, the screw could irritate or even pierce the esophagus, resulting in pain, infection, and/or possible death of the patient. Consequently, the present exemplary system and method provide an orthopedic plate system including a bone plate with thru-bores. According to the exemplary embodiments disclosed below, the exit diameter of the thru-bores may be selectively modified to secure one or more bone screws with in the thru-bores, thereby preventing the bone screws from backing out.

Moreover, the present exemplary system and method provides anti-back out protection via an integral or immediately coupled component of the bone plate. Consequently, anti-back out protection is provided independent of head height and other features of the bone screw.

In the following description, certain specific details are set forth in order to provide a thorough understanding of various embodiments of the present orthopedic plate system and method. However, one skilled in the relevant art will recognize that the present exemplary system and method may be practiced without one or more of these specific details, or with other methods, components, materials, etc. In other instances, well-known structures associated with orthopedic plate systems have not been shown or described in detail to avoid unnecessarily obscuring descriptions of the present exemplary embodiments.

Unless the context dictates otherwise, throughout the specification and claims which follow, the word “comprise” and variations thereof, such as, “comprises” and “comprising” are to be construed in an open, inclusive sense, that is, as “including, but not limited to.”

The term “compliant mechanisms” relates to a family of devices in which integrally formed flexural members provide motion through deflection. Such flexural members may therefore be used to replace conventional multi-part elements such as pin joints. Compliant mechanisms provide several benefits, including backlash-free, wear-free, and friction-free operation. Moreover, compliant mechanisms significantly reduce manufacturing time and cost. Compliant mechanisms can replace many conventional devices to improve functional characteristics and decrease manufacturing costs. Assembly may, in some cases, be obviated entirely because compliant structures often consist of a single piece of material.

Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment. Furthermore, the particular features, structures, or characteristics may be combined in any suitable manner in one or more embodiments.

### Exemplary Structure

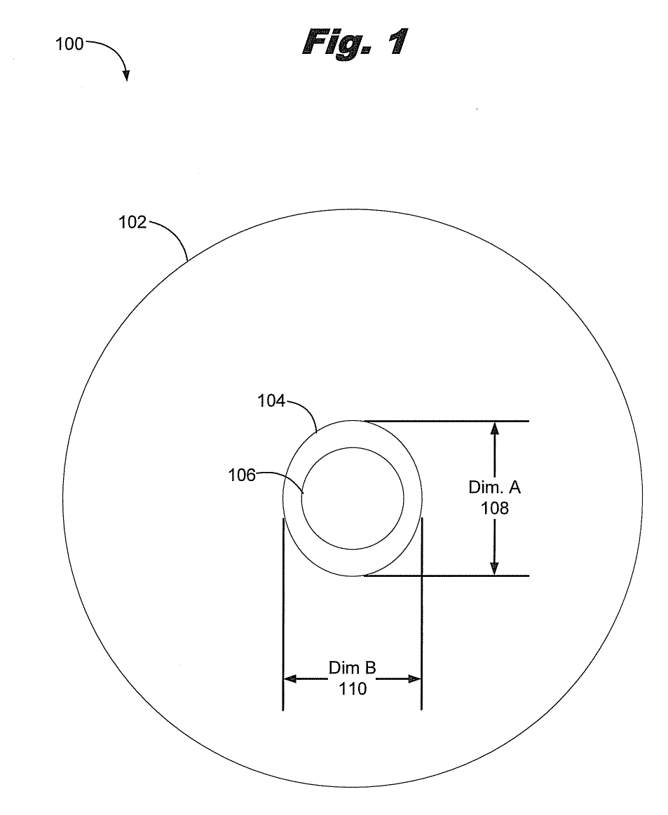

FIG. 1 is an illustrative depiction (100) of a top view of a portion of an exemplary orthopedic plate (102) with a thru-bore (106), according to one exemplary embodiment. As illustrated, the figure shows a circular portion of an orthopedic plate (102) configured to be secured to bone tissue. The orthopedic plate (102) is in no way limited to the circular shape shown; it could be any shape to better fit its exact placement inside a patient.

In the middle of the illustrated orthopedic plate (102) is a thru-bore (106) configured for a screw to be inserted therein. According to one exemplary embodiment, the thru-bore contains an upper plane and a lower plane. The upper plane is an oval shaped opening (104) that surrounds the thru-bore (106). The lower plane is an undercut groove with a larger diameter than the oval shaped opening in the upper plane. In the case of the figure, the vertical dimension, diameter A (108) is larger than the horizontal dimension, diameter B (110). The precise ratio of diameter A (108) to diameter B (110) may vary slightly through different embodiments.

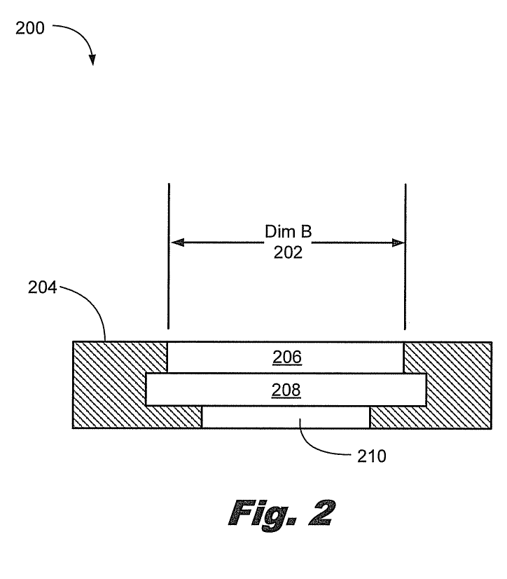

FIG. 2 is an illustrative depiction (200) of a side view of an exemplary thru-bore (210) in an orthopedic plate (204), according to one exemplary embodiment. As illustrated in the side view of FIG. 2, the upper plane includes an oval shaped rim (206) undercut by a lower plane containing a groove (208) cut deeper into the orthopedic plate (204). The groove (208) formed in the lower plane may, according to one exemplary embodiment, have the same shape as the above oval rim (206) but with a greater diameter. Alternatively, in one embodiment, the groove (208) may have a circular shape as opposed to an oval shape. Regardless of the shape of the undercut groove, the inclusion of an undercut feature facilitates the secure placement of a positionable element to be placed inside the thru-bore. That is, according to one exemplary embodiment described in further detail below, the undercut groove creates a channel wherein a positionable element may be securely positioned without the likelihood of unintentional removal.

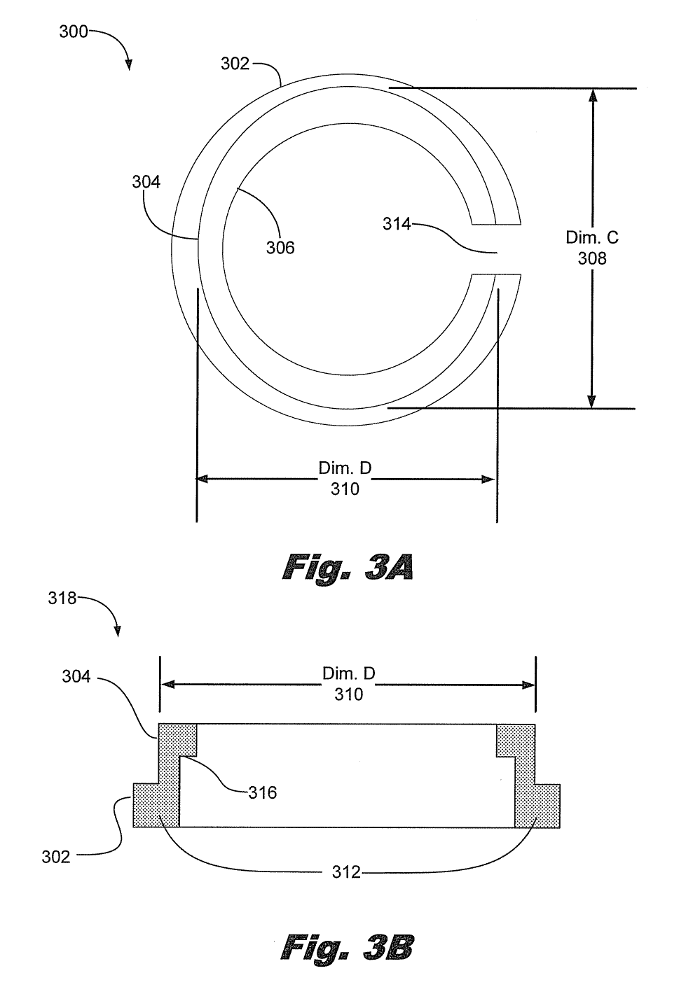

FIG. 3A is an illustrative depiction (300) of a top view of an exemplary positionable element, according to one embodiment. In one embodiment, the positionable element is a compressible retention member. As illustrated in FIG. 3A, the compressible retention member (306) is designed to fit into the thru-bore described in FIG. 1 and FIG. 2 via a number of features formed on the compressible retention member. As shown, the compressible retention member includes an upper plane with an outer edge (304) above a lower plane (302) which extends farther than the outer edge (304). The extended lower edge (302) is configured to fit into the groove (208, FIG. 2) shown in the side view of the orthopedic plate (204, FIG. 2). As illustrated in FIG. 3A, the outer edge (304) of the upper plane is oval shaped with the vertical dimension, diameter C (308) being greater than the horizontal dimension, diameter D (310). The precise ratio of diameter C (308) to diameter D (310) may vary slightly through different embodiments. The lower edge (302) may be either an oval shape to match the upper edge (304), or another shape including, but in no way limited to, a circular shape. The exemplary compressible retention member (306) illustrated in FIG. 3A includes a ring body having a gap (314) so as to allow the ring to contract and reduce the size of its diameter when an adequate force is imparted thereon. The compressible retention member (306) illustrated in FIG. 3A may be made of any appropriate material that will allow the compressible retention member to sufficiently flex to close the gap (314) without plastically deforming and/or failing while having sufficient structural properties to retain a bone screw in an associated plate, including, but in no way limited to, titanium, stainless steel, and the like

FIG. 3B is an illustrative depiction (318) of a cross-sectional side view of a compressible retention member (306), according to one exemplary embodiment. FIG. 3B illustrates how the lower plane edge (302) of the compressible retention member (306) extends beyond the upper plane edge (304) of the compressible retention member. According to one exemplary embodiment, the lower section may be referred to as the flange (312) or an engagement flange. According to the exemplary embodiment illustrated in FIG. 3B, the flange (312) is designed to fit into the lower plane groove (208, FIG. 2) of an orthopedic plate (204, FIG. 2) in order to selectively retain the compressible retention member (306) in the orifice of the orthopedic plate. While the present exemplary embodiment of the compressible retention member is described, for ease of illustration, as having a flange (312) that is designed to fit into the lower plane groove of an orifice for securing the compressible retention member, any number of fixation systems configured to retain the ring in the orifice prior to its constriction may be implemented including, but in no way limited to, a hinged member, a machined protrusion formed on the plate itself, an adhesive, or the like.

FIG. 3B further illustrates the exemplary features formed on the inner surface of the compressible retention member (306), according to one exemplary embodiment. As illustrated, the compressible retention member (306) may be formed so as to form an overhang (316) protruding into the inner diameter of the compressible retention member. According to one exemplary embodiment, when the compressible retention member (306) receives a bone screw including a head portion that is inserted past the overhang (316), the overhang may be selectively translated over a portion of the head of a bone screw by compression of the compressible retention member. Once compressed, the overhang of the compressible retention member (306) will cover up the head portion of the bone screw after the screw has been inserted into the orthopedic plate (204, FIG. 2), thereby preventing back-out of the bone screw. While the overhang is illustrated as being a single solid protrusion, any number of independent protrusions may be formed on the compressible retention member to prevent back-out of the bone screw after insertion.

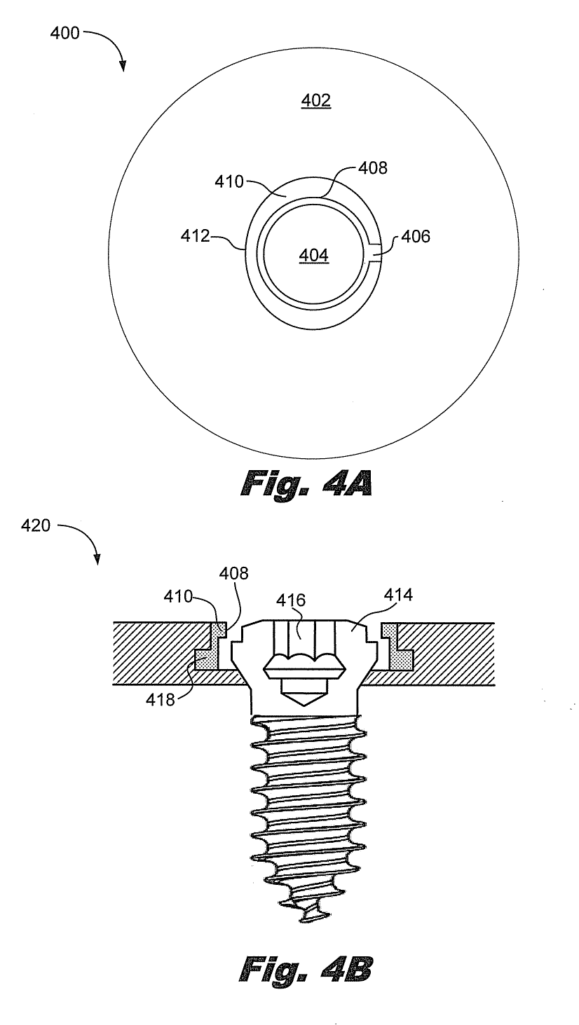

Continuing with the present exemplary configuration, FIG. 4A is an illustrative depiction (400) of a top view of an exemplary orthopedic plate (402) with an exemplary compressible retention member (410) placed inside the thru-bore (404). Specifically, FIG. 4A illustrates the orthopedic plate as shown in FIG. 1 with the compressible retention member (410) as shown in FIG. 3A placed to fit with the flange (312, FIG. 3A) fit into the groove (208, FIG. 2). In the initial position, before the screw has been inserted and driven into place, the compressible retention member (410) is positioned so that the larger dimension (308, FIG. 3A) of the oval shaped edge of the compressible retention member (304, FIG. 3A) is aligned with the larger dimension (108, FIG. 1) of the oval rim (104, FIG. 1). In this illustrated position, the screw is able to pass through the compressible retention member (410) and be driven into through the thru-bore (404) substantially un-obstructed. Once inserted, the oval shaped compressible retention member (304) can be actuated to retain the screw and prevent unintentional back-out of the screw.

In FIG. 4A, the orthopedic plate (402) is illustrated with a thru-bore (404) defined therein. From the top view of FIG. 4A, the upper edge of the compressible retention member (410) is illustrated as being disposed into the upper plane oval rim (412) of the orthopedic plate (402). The inner edge (408) of the compressible retention member (410) is configured to be sized wide enough to allow a screw, and particularly a screw head, to pass through and be driven through the thru-bore (404) into the targeted bone tissue. According to the exemplary embodiment illustrated in FIG. 4A, the gap (406) formed on the compressible retention member (410) is aligned on a side with the smaller radius of the oval. However, the gap (406) may alternatively be disposed on any portion of the oval to provide differing compression properties.

FIG. 4B is an illustrative depiction (420) of a cross-sectional side view of an exemplary orthopedic plate with an exemplary compressible retention member (410) and screw (414) disposed inside the thru-bore (404). FIG. 4B clearly illustrates how the inner edge (408) of the compressible retention member (410) is wide enough to allow the screw (414) to pass there through and be driven into place. As illustrated, the flange (418) is placed to fit into the undercut groove (208, FIG. 2) and maintain the position of the compressible retention member (410) in the plate. According to the present exemplary system and method, the screw can have any type of screw head (416) as compression of the compressible retention member (410) will create interference with the screw head (416) to prevent the screw from backing out after insertion. According to one exemplary embodiment, the screw head slot is a hex shape and may include features configured to enhance an engagement between the compressed compressible retention member (410) and the screw head (416). Engagement features formed on the screw head (416) may include, but are in no way limited to, recesses, channels, and the like.

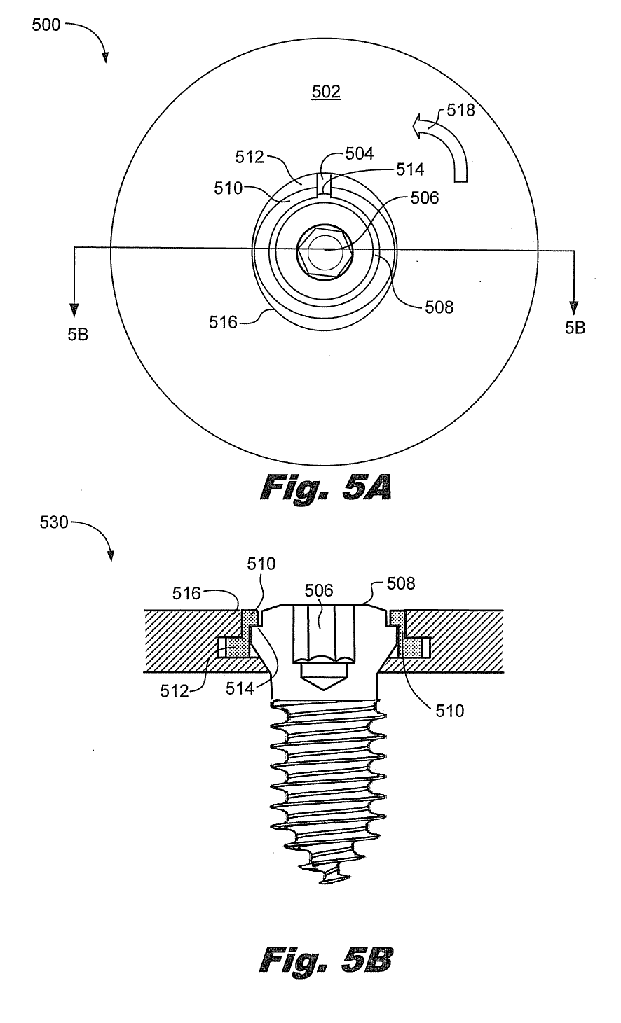

FIG. 5A is an illustrative depiction (500) of a top view of an exemplary orthopedic plate (502) with an exemplary secured compressible retention member (510) and screw (506) inside the thru-bore once the exemplary screw has been inserted. According to one exemplary embodiment, once the screw (506) has been driven into place through the thru-bore, the compressible retention member (510) is secured by rotating (518) the compressible retention member such that the lobed portions of the compressible retention member interfere with and are compressed by the inner surface of the oval shaped rim (516) corresponding to the oval shaped rim having a reduced radius. When rotated, the interference between the lobed portions of the compressible retention member and the smaller dimension of the oval shaped rim (516) cause the compressible retention member (510) to compress and reduce its effective diameter. With the compressible retention member (510) in this position, the gap (504) will become smaller as the larger dimension of the compressible retention member (510) is pressed into the smaller dimension of the oval rim (516). With the compressible retention member (510) is this position, the overhang will cover part of the top edge on a screw (508). By covering over the edge, the screw is prevented from unintentionally backing out of position. Furthermore, according to the present exemplary embodiment, the above-mentioned configuration allows the flange (512) to fit into the groove (208, FIG. 2) of the orifice, causing the flange (512) to prevent the compressible retention member (510) from popping out of the orifice if a backing force is applied to the compressible retention member (510) by the head of the screw.

FIG. 5B is an illustrative depiction (530) of a cross-sectional side view of the exemplary orthopedic plate (502) system of FIG. 5A with an exemplary secured compressible retention member (510) and screw (508) inside the thru-bore, taken along the line 5B. The cross-sectional side view of FIG. 5A illustrates one exemplary embodiment of how the present exemplary compressible retention member (510) can be compressed closer to the screw, via an interference between the lobed portions of the compressible retention member and the inner surface of the oval orifice, so that the overhang (514) covers a part of the top edge of a screw (508). With the compressible retention member (510) compressed to the exemplary position illustrated in FIG. 5B, there is now no space between the compressible retention member (510) and the edge of the oval rim (516), thereby providing a maintaining force or the compressible retention member to maintain the position of the inserted screw (508). The side view shows how the flange (512) is still within the groove (208, FIG. 2) so as to prevent the compressible retention member (510) from popping out. Like in other figures, the screw head (506) is not limited to the hex shape depicted in the figure.

While the present exemplary system has been described herein as a bone plate system including a body defining at least one thru-bore with a central cavity, the central cavity including a compressible member being configured to modify a top exit diameter of the thru-bore, a number of variations on the configuration and position of the compressible member configured to modify a portion of the diameter of the thru-bore may be made. Specifically, according to one exemplary embodiment, the compressible member may, according to one alternative embodiment, reside entirely within the thru-bore. According to this exemplary embodiment, non-circular features may be formed on the walls of the thru-bore or on the screw head (506) itself to engage and either compress or expand the compressible retention member. According to one exemplary embodiment, the engagement and increased compression of the compressible retention member may be actuated by a rotation that is opposite the insertion rotation of the bone screw. Back-out of a screw is a reverse rotation phenomenon. Consequently, according to this exemplary embodiment, by designing the actuation and increased compression of the compressible retention member (510) to be via rotation opposite the insertion of the bone screw, any reverse rotation of the bone screw that does occur will cause the compressible retention member to further actuate and engage, assuring retention of the bone screw.

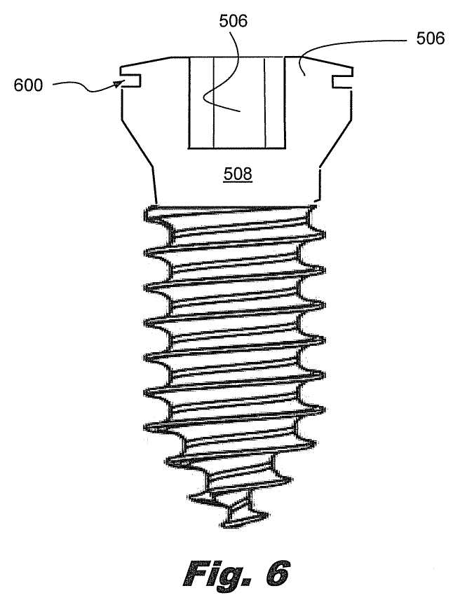

Additionally, according to various exemplary embodiments, the retention interface maintaining the bone screw may be caused by the engagement of non-circular surfaces of any number of parts. Specifically, according to one exemplary embodiment, the non-circular interface may occur between the screw head (506) and the inner surface of the thru-bore, between the compressible retention ring and the screw head itself, and the like. According to one alternative embodiment, the compressible retention member could be fixed to the screw head. As illustrated in FIG. 6, a circumferential groove (600) may be formed in the head portion (506) of the bone screw (508) to receive a compressible retention member. According to one exemplary embodiment, the inner surface of the circumferential groove (600) may have a non-circular diameter correlating with a non-circular inner surface diameter of the associated compressible retention member. According to this exemplary embodiment, an engagement feature (not shown) may also be formed in the thru-bore to engage the compressible retention member when the bone screw/retention member combination has been sufficiently inserted. Once engaged, the engagement member may prevent further rotation of the compressible retention member as the screw is further advanced. According to this exemplary embodiment, further rotation of the bone screw (508) will cause an interference between the non-circular inner surface of the circumferential groove (600) and the non-circular inner surface diameter of the associated compressible retention member, causing the compressible retention member to expand and further engage the inner surface of the thru-bore, resulting in retention of the bone screw. As noted, the present exemplary system and method may be implemented in any number of alternative configurations.

In sum, the present exemplary orthopedic plate and associated fastening system includes an exemplary orthopedic plate having a thru-bore with an oval shaped rim and an internal groove underneath disposed adjacent to the rim. A compressible retention member configured to mate with the thru-bore of the orthopedic plate includes, according to one exemplary embodiment, an upper plane and a lower plane. According to one exemplary embodiment, the upper plane configured to interact with the oval shaped rim of the thru-bore is defined by an oval perimeter having a maximum diameter smaller than a maximum diameter of the lower plane. Additionally, according to the exemplary embodiment, the lower plane is configured to engage the internal groove of the thru-bore of the orthopedic plate to maintain the mechanical engagement between the compressible retention member and the orthopedic plate during operation. Once assembled, a bone screw may be passed through the thru-bore and the associated compressible retention member. After the screw has been inserted, the compressible retention member may be rotated, initiating an interference contact between the oval shaped rim of the thru-bore and the upper plane of the compressible retention member, resulting in a compression of the larger dimension of the oval shaped ring into the smaller dimension of the oval shaped rim. Reduction of the effective diameter of the compressible retention member positions an overhang section above at least a portion of the screw to prevent the screw from unintentionally backing out from a secured inserted position

While the present exemplary rotationally locking cervical plate system has been described, for ease of explanation only, in the context of a cervical plate system, the present exemplary systems and methods may be applied to any number of orthopedic fixtures. Specifically, the present screw back out prevention components may be used to couple any number of orthopedic apparatuses to a desired bone, for any number of purposes, as long as the connecting orthopedic apparatus includes a thru-bore substantially conforming to the configurations described herein.

In conclusion, the present exemplary systems and methods provide for coupling an orthopedic plate to one or more bones while preventing back-out of the fastener.

The preceding description has been presented only to illustrate and describe the present method and system. It is not intended to be exhaustive or to limit the present system and method to any precise form disclosed. Many modifications and variations are possible in light of the above teaching.

The foregoing embodiments were chosen and described in order to illustrate principles of the system and method as well as some practical applications. The preceding description enables others skilled in the art to utilize the method and system in various embodiments and with various modifications as are suited to the particular use contemplated. It is intended that the scope of the present exemplary system and method be defined by the following claims.

1. An orthopedic device comprising;
an implant member defining at least one thru-bore, wherein said thru-bore is defined as including an upper non-circular rim member having a maximum diameter and an internal groove defined adjacent to said non-circular rim member, said internal groove having a maximum diameter; and
a compressible retention member configured to be disposed in said thru-bore, said compressible retention member including a top plane and a bottom plane;
wherein said top plane of said compressible retention member has a non-circular perimeter surface configured to engage said upper non-circular rim member of said thru-bore; and
wherein said bottom plane of said compressible retention member is configured to rotatably engage said internal groove of said thru-bore;
wherein a rotation of said compressible retention member within said thru-bore reduces an effective diameter of said thru-bore.

2. The orthopedic device of claim 1, further comprising at least one bone screw being configured to be fastened to a bone segment through said thru-bore;
wherein said bone screw has a head portion defined by maximum diameter;
wherein said effective diameter of said thru-bore when said compressible retention member is compressed is less than said maximum diameter of said head portion of said bone screw.

3. The orthopedic device of claim 2, wherein said thru-bore is further defined by an exit orifice;
wherein said exit orifice has a diameter less than said maximum diameter of said head portion of said bone screw.

4. The orthopedic device of claim 3, wherein said upper non-circular rim member of said thru-bore is oval in shape.

5. The orthopedic device of claim 3, wherein said maximum diameter of said upper non-circular rim member is smaller than said maximum diameter of said internal groove.

6. The orthopedic device of claim 3, wherein said non-circular perimeter surface of said top plane of said compressible retention member comprises an oval shape.

7. The orthopedic device of claim 1, wherein said compressible retention member comprises a split-ring.

8. The orthopedic device of claim 1, wherein said compressible retention member comprises an integral compliant member formed on said implant member

9. The orthopedic device of claim 1, wherein said compressible retention member further comprises at least one protrusion extending away from said perimeter in said top plane.

10. The orthopedic device of claim 9, wherein said at least one protrusion extending away from said perimeter in said top plane comprises an overhang.

11. An orthopedic device comprising;
an implant member defining at least one thru-bore, wherein said thru-bore is defined as including an upper oval rim member having a maximum diameter and an internal groove defined adjacent to said oval rim member, said internal groove having a maximum diameter; and
a compressible retention member configured to be disposed in said thru-bore, said compressible retention member including a top plane and a bottom plane;
wherein said top plane of said compressible retention member has oval perimeter surface configured to engage said upper oval rim member of said thru-bore; and
wherein said bottom plane of said compressible retention member is configured to rotatably engage said internal groove of said thru-bore;
wherein a rotation of said compressible retention member within said thru-bore reduces an effective diameter of said thru-bore.

12. The orthopedic device of claim 11, further comprising at least one bone screw being configured to be fastened to a bone segment through said thru-bore;
wherein said bone screw has a head portion defined by maximum diameter;
wherein said effective diameter of said thru-bore when said compressible retention member is compressed is less than said maximum diameter of said head portion of said bone screw.

13. The orthopedic device of claim 12, wherein said thru-bore is further defined by an exit orifice;
wherein said exit orifice has a diameter less than said maximum diameter of said head portion of said bone screw.

14. The orthopedic device of claim 11, wherein said compressible retention member comprises a split-ring.

15. The orthopedic device of claim 11, wherein said compressible retention member comprises an integral compliant member formed on said implant member

16. The orthopedic device of claim 11, wherein said compressible retention member further comprises at least one protrusion extending away from said perimeter in said top plane.

17. The orthopedic device of claim 16, wherein said at least one protrusion extending away from said perimeter in said top plane comprises an overhang.

18. An orthopedic device comprising;
an implant member defining at least one thru-bore, wherein said thru-bore is defined as including a non-circular feature on an inner surface of said thru-bore, said non-circular feature having a maximum diameter; and
a compressible retention member configured to be disposed in said thru-bore, said compressible retention member including a non-circular perimeter surface configured to engage said non-circular feature of said thru-bore; and
wherein a rotation of said compressible retention member within said thru-bore reduces an effective diameter of said thru-bore.

19. The orthopedic device of claim 18, further comprising at least one bone screw being configured to be fastened to a bone segment through said thru-bore;
wherein said bone screw has a head portion defined by maximum diameter;
wherein said effective diameter of said thru-bore when said compressible retention member is compressed is less than said maximum diameter of said head portion of said bone screw.

20. The orthopedic device of claim 19, wherein said thru-bore is further defined by an exit orifice;
wherein said exit orifice has a diameter less than said maximum diameter of said head portion of said bone screw.
